(19) United States
(12) Patent Application Publication
(10) Pub. No.: US 2005/0222205 A1
(43) Pub. Date: Oct. 6, 2005

(54) Treatment of pain with ifendropil

(76) Inventors: John Brew, Saffron Walden, Essex (GB); Robin Mark Bannister, Essex (GB); Andrew Douglas Baxter, Essex (GB); Alan Rothaul, Essex (GB); Michael Harvey Lyne, Essex (GB)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/512,893

(22) PCT Filed: May 6, 2003
(86) PCT No.: PCT/GB03/01906

§ 371 Date: Oct. 28, 2004

(30) Foreign Application Priority Data
May 3, 2002 (GB) 0210264.8

(51) Int. Cl. A61K 31/445

(52) U.S. Cl. 514/317

(57) ABSTRACT

Ifenprodil is useful for the treatment of pain, e.g. on administration intranasally or by another route that avoids first-pass metabolism.

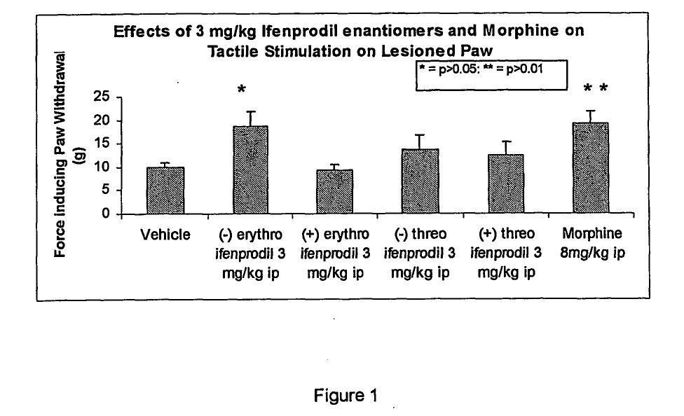

### FIELD OF THE INVENTION

This invention relates to the use of a known compound for the treatment of pain.

### BACKGROUND OF THE INVENTION

N-methyl-D-aspartate (NMDA) receptor antagonists have been long known to exhibit anti-nociceptive effects, and a number have proven efficacy in the treatment of a number of neuropathies, including postherpetic neuralgia, central pain caused by spinal cord injury and phantom limb pain. The NMDA receptor antagonist dextrorphan is disclosed for the treatment of pain in EP-A-0615749 and also, along with a number of other such compounds (including ifenprodil), in WO-A-97/14415. Unfortunately, most agents which block the NMDA receptor also induce unacceptable side-effects at analgesic doses, including memory impairment, ataxia, hallucinations and dysphoria, which prohibit their widespread use.

Ifenprodil, i.e. 2-(4-benzylpiperidino)-1-(4-hydroxyphenyl)-1-propanol, selectively blocks NR2B-containing NMDA receptors in a voltage-independent and non-competitive manner (Gallagher et al., 1996, J. Biol. Chem. 271(16):9603-9611) and exhibits anti-nociceptive activity in animal models of acute and chronic pain (Taniguchi et al., 1997, Brit. J. Pharmacol. 122, 809-812; Boyce et al., 1999, Neuropharmacology 38:611-623). Ifenprodil (as ifenprodil tartrate) is commercially available as a racemic mixture of the erythro diastereomer.

Ifenprodil also exhibits potent alpha-1 adrenergic receptor binding properties (Chenard et al., 1991, J. Med. Chem. 34 (10):3085-3090) which can cause hypotension and syncope in some recipients. It is also reported by Chenard et al. that the threo isomers of ifenprodil have selectivity for the NMDA receptor over the alpha-1 adrenoreceptor.

### SUMMARY OF THE INVENTION

The present invention is based on the discovery that ifenprodil has utility in the treatment of pain, including neuropathic pain and migraine headache.

As suggested above, it has been thought in the past that (−)-threo ifenprodil has improved NMDA activity over the other enantiomers, and that this would produce a significant improvement in efficacy. Surprisingly, it has now been demonstrated that (−)-threo ifenprodil does not produce a significant increase in efficacy over the other enantiomers in a model of neuropathic pain. However, it has been shown that this enantiomer has lower hypotension liability (alpha1-adrenoceptor antagonism), which will improve its side-effect profile over the other enantiomers.

### DESCRIPTION OF PREFERRED EMBODIMENTS

Ifenprodil has two chiral centres. Any reference herein to ifenprodil should be understood as a reference to any enantiomer or mixture thereof. Any enantiomer may be substantially free of others, e.g. in an enantiomeric excess of at least 80%, preferably at least 90% and more preferably at least 95%. Similarly, any mixture of diastereomers may be substantially free of the other. The threo form, and in particular the (−)-threo form, may be preferred in certain cases; the (−)-erythro form may be preferred in others.

The ifenprodil may be in the form of the free base or any pharmaceutically acceptable salt, e.g. the tartrate, or in the form of a metabolite or prodrug. Such forms are known to those of ordinary skill in the art.

The active agent may be administered by, for example, the oral, topical, dermal, ocular, intravenous, intraarticular, rectal, vaginal, inhalation, intranasal, sublingual or buccal route. The amount of active ingredient that is used can be chosen by the skilled person having regard to the usual factors.

For use, the active agent is typically formulated, e.g. with a conventional diluent or carrier, or as a patch, as a medicament adapted to be delivered by the chosen route. Such formulations are known to those skilled in the art, and will be chosen according to the usual considerations such as the potency of the drug, the severity of the condition and the route of administration.

Ifenprodil is preferably administered intranasally, buccally or by any route that avoids first-pass metabolism. Indeed, nasal delivery introduces significant concentrations of ifenprodil and its isomers to NMDA receptors whilst reducing side-effects caused by the unwanted alpha-1 adrenoreceptor-binding activity. In this context, a typical daily dose is less than 60 mg, e.g. 1 to 50 mg, ifenprodil; a higher dose, e.g. up to 500 mg, may be used, especially if first-pass metabolism is not avoided.

In particular, it would be of benefit to administer ifenprodil in a manner that reduced peripheral exposure to vascular smooth muscle (minimise effect on vascular tone), while maximising the concentrations in the CNS (maximise analgesia). This may be done by nasal delivery, reducing systemic load, while maximising the concentration of drug in the CNS. By way of example only, a composition for intranasal delivery comprises, in addition to ifenprodil, one or more of a solubility enhancer such as propylene glycol, a humectant such as mannitol, a buffer and water. A mucoadhesive agent may also be used.

Ifenprodil has very poor pharmacokinetics, with very high first-pass metabolism (5% bioavailability and a short half life; t1/2 1 hour). Consequently, administering ifenprodil orally, to treat a chronic condition like neuropathic pain, may require high and frequent doses. Dermal administration, e.g. by the use of a dermal patch, allows chronic dosing of this compound, while avoiding first-pass metabolism and so lowering the dose. Additionally, there is the potential of removing the dose from the circulation rapidly at the end of the treatment period.

It will often be advantageous to use ifenprodil in combination with another drug used for pain therapy. Such another drug may be an opiate or a non-opiate such as baclofen. Especially for the treatment of neuropathic pain, coadministration with gabapentin is preferred.

The following experiments provide the evidence on which the present invention is based.

In a test on the effect of agents on thermal stimulation of the lesioned paw, ifenprodil (at 3, 10 and 30 mg/kg ip) was more effective than gabapentin (at 30 mg/kg ip) and almost as effective as morphine (at 8 mg/kg ip). Such tests showed that (−)-threo-ifenprodil was the most effective enantiomer (at 3 mg/kg ip).

Evidence of the incorrect conclusion reached by Chenard et al., 1991, supra, is provided by experiments conducted in a Bennett model (Bennett et al, 1988, Pain 33(1):87-107). The results are shown in the accompanying drawing.

The following Example illustrates a composition suitable for intranasal delivery. In this Example, 1-10 mg ifenprodil is included in 100 μl of:

| Excipient: | % w/w | |
|---|---|---|
| Benzalkonium chloride | 0.02 | Preservative |
| Propylene Glycol | 25 | Solubility Enhancer |
| Mannitol | 15 | Humectant |
| HNa₂PO₄ (0.2 M) | 25.2 | |
| Citric Acid (0.1 M) | 10.0 | |
| Deionised water | 24.6 | (pH 6.5 buffer) |

1. A method for treating pain wherein said method comprises administering, to a patient in need of such treatment, an effective amount of ifenprodil.

2. The method, according to claim 1, for the treatment of neuropathic pain.

3. The method, according to claim 1, for the treatment of migraine.

4. The method, according to claim 1, wherein the ifenprodil is in the form of either or both threo enantiomers.

5. The method, according to claim 4, wherein the ifenprodil is (−)-threo-ifenprodil.

6. The method, according to claim 1, wherein the ifenprodil is (−)-erythro-ifenprodil.

7. The method, according to claim 1, wherein said ifenprodil is administered via a route that avoids first-pass metabolism.

8. The method, according to claim 6, wherein the route is intranasal.

9. The method, according to claim 6, wherein the route is dermal.

10. The method, according to claim 7, wherein less than 60 mg ifenprodil is administered.

11. A composition, suitable for intranasal delivery, which comprises an aqueous solution of ifenprodil, a solubility enhancer and a humectant.

12. The composition according to claim 11, wherein the ifenprodil is (−)-threo-ifenprodil.

13. The composition according to claim 11, wherein the ifenprodil is (−)-erythro-ifenprodil.

14. The composition, according to claim 1, wherein the ifenprodil is threo-ifenprodil and the medicament is formulated for administration via a route that avoids first-pass metabolism.

15. The composition, according to claim 14, wherein the ifenprodil is (−)-threo-infenprodil.
